(19) United States
(12) Patent Application Publication
(10) Pub. No.: US 2022/0362136 A1
(43) Pub. Date: Nov. 17, 2022

(54) USE OF EXTRACT OF AQUILARIA MALACCENSIS SEED IN PREPARATION OF SKIN CARE COMPOSITION

(71) Applicants: Chen HSIEH, Kaohsiung City (TW); Hui-Ping Hsieh, Kaohsiung City (TW)

(72) Inventors: Chen HSIEH, Kaohsiung City (TW); Hui-Ping Hsieh, Kaohsiung City (TW)

(21) Appl. No.: 17/316,739

(22) Filed: May 11, 2021

(51) Int. Cl. A61K 8/9789; A61Q 19/00

(52) U.S. Cl. CPC A61K 8/9789; A61Q 19/00; A61K 2800/10; A61K 2800/82; A61Q 19/08

(57) ABSTRACT

A Aquilaria malaccensis seed extract for use in preparation of skin care composition is provided. Multiple Aquilaria malaccensis seeds are washed for three times and drained to dry in room temperature, and are then spread on a water-absorbing material for drying in shade. The Aquilaria malaccensis seeds so dried are pulverized to a particle size not greater than 2 mm. The pulverized Aquilaria malaccensis seeds are subject to ultrasonic extraction with 90% ethanol in such a way that the pulverized Aquilaria malaccensis seeds are subjected to extraction for three times, one time for each day. After the completion of the extraction, all ethanol-water solvent is removed to obtain Aquilaria malaccensis seed extract (Crude-EtOH).

### TECHNICAL FIELD OF THE INVENTION

The present invention relates to a use of extract of Aquilaria malaccensis seed in preparation of a skin care composition

### DESCRIPTION OF THE PRIOR ART

Human skin undergoes certain phenomena, such as aging, roughening, or wrinkling due to reasons of age and physiology or influence of the environment. Normal skin of young people generally possesses elasticity and tension, and when mimetic muscles get relaxed, the skin recovers quickly and wrinkle become vanishing. However, upon entry middle or old ages, the skin suffers aging phenomena, such as thinning, drying, and tension lowering, so that when the mimetic muscles relax, the skin does no recover quickly and wrinkles start to form. Also, the skin may be affected by various factors, such as drying, ultraviolet light, detergent, or chemicals or imbalance of hormones, and also due to lowering of protection of skin horny layer and water contents, to become roughening and lose elasticity and wet-keeping function, and consequentially, the skin become wrinkled and dull.

Although various skin care and treatment products are currently available in the market for skin quality improving and aging resisting, such products are generally made primarily up of chemicals, and due to such non-natural ingredients, they may easily cause allergy or may provide only insignificant effect due to individual physique factors, or may even cause severer damage to the skin. Thus, it is an important challenge to develop a natural ingredient based product that treats damaged skin and enhances caring of skin.

### SUMMARY OF THE INVENTION

An objective of the present invention is to overcome the above-discussed drawbacks by providing an extract of Aquilaria malaccensis seed that can be used to prepare a skin treatment and care composition.

To achieve the above objective, the present invention provides an Aquilaria malaccensis seed extract for use in preparation of a skin care composition, wherein multiple Aquilaria malaccensis seeds are subjected to wash, in separate batches, with flowing reverse osmosis water; each wash is performed for 10 minutes; each batch of the Aquilaria malaccensis seeds is washed repeatedly for three times, and is then placed in room temperature for draining to dry, and is then spread and laid on a water-absorbing material for drying in shade for two days; and afterwards, a pulverizing device is used to pulverize the Aquilaria malaccensis seeds, such that the pulverized Aquilaria malaccensis seeds have a particle size that is not greater than 2 mm; and the pulverizing device is controlled in respect of pulverization time such that continuous pulverization is performed only for a time period not exceeding 20 minutes; next, the pulverized Aquilaria malaccensis seeds are subject to ultrasonic extraction, in separate batches, with 90% ethanol in such a way that each batch of the pulverized Aquilaria malaccensis seeds is subjected to extraction for three times, with only one extraction performed in each day; and after the completion of extraction, all ethanol-water solvent is removed by means of a rotary evaporator and a vacuum pump, so as to obtain Aquilaria malaccensis seed extract.

As such, the Aquilaria malaccensis seed extract provides a use for preparation of a skin care and repair composition.

### BRIEF DESCRIPTION OF THE DRAWINGS

FIG. 1 is a flow chart illustrating steps of making an extract of Aquilaria malaccensis seed according to the present invention.

FIG. 2 is a graph of inspection of the Aquilaria malaccensis seed extract according to the preset invention after being kept in a refrigerated and light-blocked condition for one month.

FIG. 3 is a graph of cell viability for the Aquilaria malaccensis seed extract according to the preset invention after being subjected ultraviolet light.

FIG. 4 is photos showing performance of the Aquilaria malaccensis seed extract according to the preset invention for detecting UVB photoproduct cyclobutene pyrimidine dimer (CPD).

FIG. 5 is a graph illustrating performance of the Aquilaria malaccensis seed extract according to the preset invention for detecting UVB photoproduct CPD.

FIG. 6 is a graph illustrating test for the Aquilaria malaccensis seed extract according to the preset invention enhancing generation of skin collagen.

FIG. 7 is photos illustrating test for the Aquilaria malaccensis seed extract according to the preset invention enhancing wound healing.

FIG. 8 is a graph illustrating test for test for the Aquilaria malaccensis seed extract according to the preset invention enhancing wound healing extract according to the preset invention enhancing wound healing.

### DETAILED DESCRIPTION OF THE PREFERRED EMBODIMENT

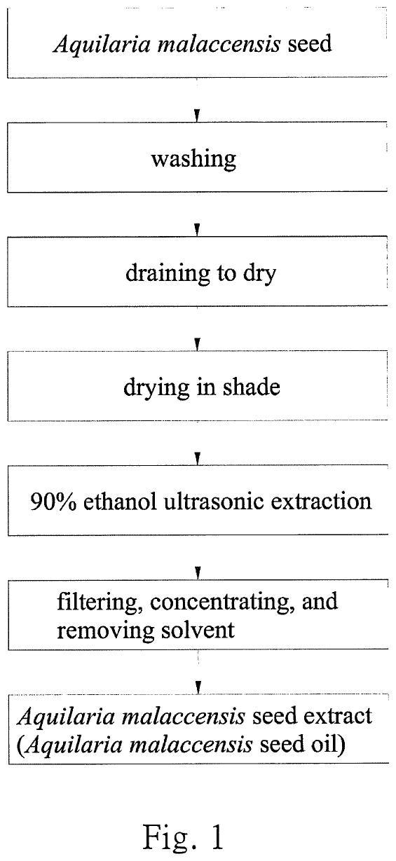

As shown in FIG. 1, multiple Aquilaria malaccensis seeds are subjected to wash, in separate batches, with flowing reverse osmosis water. Each wash is performed for 10 minutes. Each batch of the Aquilaria malaccensis seeds is washed repeatedly for three times, and is then placed in room temperature for draining to dry, and is then spread and laid on a water-absorbing material for drying in shade for two days. Afterwards, a pulverizing device is used to pulverize the Aquilaria malaccensis seeds, such that the pulverized Aquilaria malaccensis seeds have a particle size that is not greater than 2 mm. Further, the pulverizing device is controlled in respect of the pulverization time such that continuous pulverization is performed only for a time period not exceeding 20 minutes, in order to prevent the chemical components of Aquilaria malaccensis from being altered by overheating of the device. Next, the pulverized Aquilaria malaccensis seeds are subject to ultrasonic extraction, in separate batches, with 90% ethanol in such a way that each batch of the pulverized Aquilaria malaccensis seeds is subjected to extraction for three times, with only one extraction performed in each day. After the completion of extraction, all ethanol-water solvent is removed by means of a rotary evaporator and a vacuum pump, so as to obtain Aquilaria malaccensis seed extract (Crude-EtOH), and the Aquilaria malaccensis seed extract (Crude-EtOH) is in an oily form and presented as an Aquilaria malaccensis seed oil.

Following the above, a preferred embodiment is that Aquilaria malaccensis seeds in an amount of 2.9 kilograms are subjected to the above-described steps of pulverizing, draining to dry, and drying in shade, and are then subjected to extraction with 15 liters of 90% ethanol to obtain 102 grams of Aquilaria malaccensis seed extract (Crude-EtOH), meaning 102 grams of Aquilaria malaccensis seed oil.

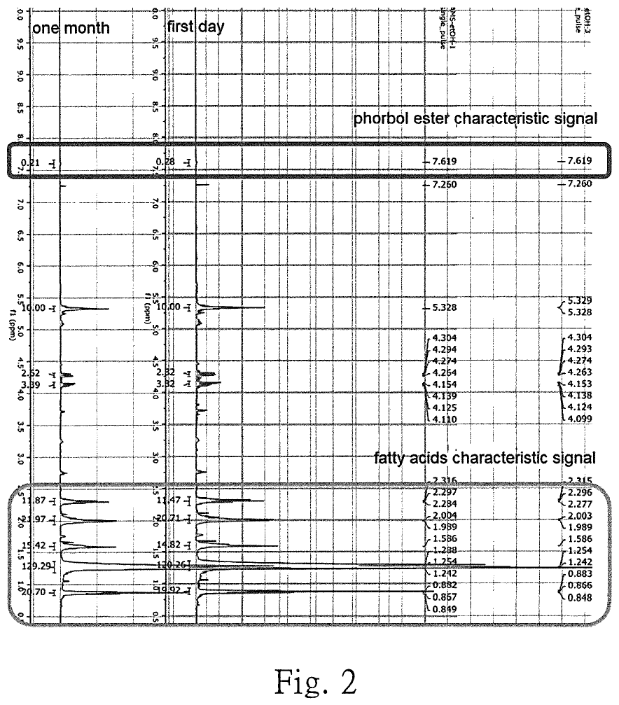

Next, the above-described Aquilaria malaccensis seed oil is stored for one month in a condition of being refrigerated at a temperature of 4° C. and shielded from light. Physical properties, such as smell, color, and pH value of the Aquilaria malaccensis seed oil do not alter. Further, evaluation is carried out with nuclear magnetic resonance (NMR) equipment, and the result is shown in FIG. 2. ¹H NMR (CDCl₃, 400 MHz) performed at the first day and the same subject of inspection after one month both show characteristic signals indicating the presence of phorbol esters and fatty acids, and no significant change in respect of the ratio of components. ¹H NMR show stable and matched result. This suggests a 4° C. refrigeration and light-blocked environment is suitable for preservation of the Aquilaria malaccensis seed oil.

Further, embodiments provided in the following experiments further evidence applications of the use of the present invention, but these are not provided to limit the scope of the present invention.

### Experiment 1

The experiment is performed due to that for any kind of product applied to the skin, such a product is brought to contact the skin horny layer first. Thus, cells of human skin keratinocyte cell line are selected for performance of safety assessment. The experiment adopts MTT assay test. Skin keratinocyte cells (1×10⁴/well) are cultivated in a 96-well plate and are cultivated in an incubator of 37° C. and 5% CO₂ for at least 24 hours. Thus, the experiment is a cell viability test for Aquilaria malaccensis seed oil protected and treated skin cells after subject to ultraviolet light.

(1) Removing cell supernatant liquid, replacing for fresh serum-free cultivation liquid, cultivating for 4 hours, and then washing with PBS and adding fresh serum-free cultivation liquid, and continuously cultivating for 4 hours, and then performing analysis of viability, this being a control group.

(2) Removing cell supernatant liquid, replacing for fresh serum-free cultivation liquid, depositing in a cell incubator for cultivating for 4 hours, removing supernatant liquid and washing with PBS and sucking to dry, subjecting to UV irradiation and then immediately adding fresh serum-free cultivation liquid to continuously cultivate for 4 hours, and then performing analysis of viability, this being a UV irradiation group.

(3) Removing cell supernatant liquid, individually mixing the Aquilaria malaccensis seed oil in fresh serum-free cultivation liquid for cultivation for 4 hours, removing supernatant liquid and washing with PBS and sucking to dry, individually subjecting to UV irradiation and then immediately adding fresh serum-free cultivation liquid that contains the extract to continuously cultivate for 4 hours, and then performing analysis of viability, this being a UV irradiation plus Aquilaria malaccensis seed oil group.

(4) Upon reaching reaction time for the above three groups, removing old cultivation liquid and washing once with PBS and replacing for fresh cultivation liquid and adding MTT (3-4,5-cimethylthiazol-2-yl)-2,5-diphenyl tetrazolium bromide) solution 10 μL for reaction, performing reaction at 37° C. and 5% CO₂ for 4 hours and then removing the cultivation liquid, adding DMSO 100 μL to dissolve formazan precipitate, and finally measuring light absorbance for wavelength 570 nm (BioTek, Synergy™2, USA).

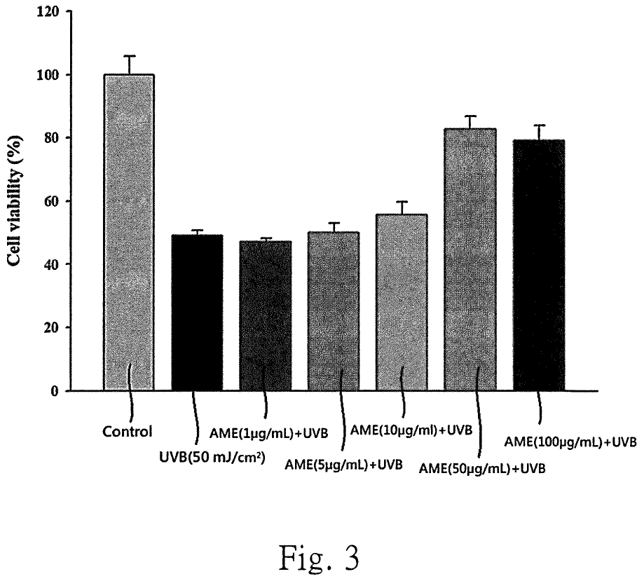

(5) Experiment results are provided in FIG. 3, which shows cell viability is around 47% when the cells are subject to irradiation of UVB at 50 MJ/cm², and if being subject to Aquilaria malaccensis seed oil of 1-100 μg/mL, damage of the cells caused by UVB can be effectively reduced, and effective protection provided by Aquilaria malaccensis seed oil of 1-100 μg/mL is most significant.

### Experiment 2

The experiment is performed due to that chemicals, radiation, and various other factors may cause damage to base pairs of DNA, and DNA requires frequent repair, of which nucleotide excision repair is one of the important methods, wherein most of DNA damages caused by ultraviolet light are excised and removed so that the cells are protected from influences caused by harmful mutation (of which the major form is cyclobutene pyrimidine dimer (CPD). Thus, the experiment is tests for the Aquilaria malaccensis seed oil protecting and treating skin cells-performance for detecting UVB photoproduct CPD.

(1) Placing 1×10⁵/ml cells in 24-well plate for cultivation for at least 24 hours, carrying to processing individually for a control group, a UV irradiation group, and a UV irradiation plus Aquilaria malaccensis seed oil group; carrying out UV irradiation after action of the Aquilaria malaccensis seed oil for 4 hours and replacing for serum-free cultivation liquid for cultivating for 4 hours; removing cultivation liquid and washing with PBS, fixing cells with ice-cold methanol, followed by action with Triton X-100, adding 2 M HCl to act for 1 hour, and filling cell gaps with 1% BSA, and applying CPD primary antibody for shaking reaction at 37° C. for 1 hour, removing the primary antibody, and then adding secondary antibody for light-blocked reaction for 30 minutes, washing with PBS and detecting fluorescence performance at Ex: 504 nm; Em: 524 nm; adding Hoechst 33342 (10 mg/ml) for nuclei staining, and washing with PBS and detecting fluorescence performance at Ex: 355 nm; Em: 460 nm, and observing and photographing with a fluorescence microscope.

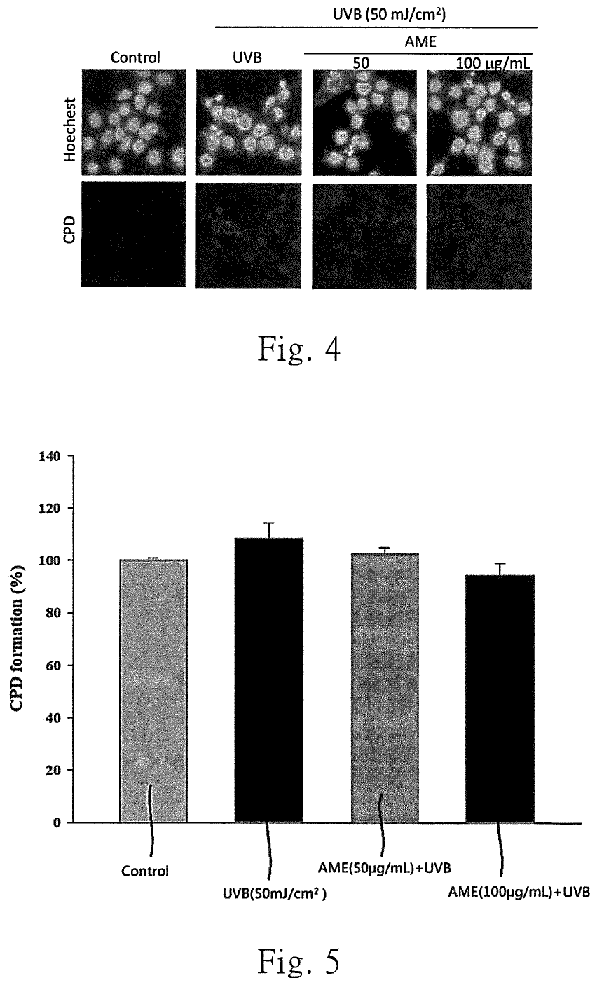

(2) Experiment results are provided in FIGS. 4 and 5, which show cells, after being irradiated with UVB, are induced to generate photoproduct CPD, and if subject to Aquilaria malaccensis seed oil of 50 and 100 μg/mL, CPD in the cells is significantly reduced, indicating the Aquilaria malaccensis seed oil provides an effect of protecting cells against damage caused by UVB.

### Experiment 3

The experiment is performed due to that skin will gradually lose collagen resulting from free radicals contained in the body, ultraviolet light, or aging, leading to loss of elasticity and tension of the skin, making early aging of the skin. The experiment is provided to inspect if the Aquilaria malaccensis seed oil can enhance generation of skin collagen.

(1) Placing 1×10⁴/ml skin fibroblast cells in a 3-cm dish plate for cultivation for at least 24 hours, removing supernatant liquid and adding serum-free cultivation liquid that contains samples for cultivation for 48 hours, washing with PBS and then scrubbing off cells, subjecting centrifugal operation at 1,200 RPM for 5 minutes, and then removing supernatant liquid. The experiment uses Sircol soluble collagen assay kit to carry out measurement of collagen. Firstly, cell sap of 100 μL is mixed with 1 mL of Sircol dye reagent, and then uniformly shaken in room temperature for 30 minute, followed by subjected to centrifugal operation at 12,000 RPM for 10 minutes, directly pouring out supernatant liquid, adding 750 μL ice-cold acid-salt wash reagent, and further subjected to centrifugal operation at 12,000 RPM for 10 minutes, and completely removing the dye reagent, and then adding 250 μL alkali reagent for uniform mixing, and picking up 1004 to place in a 96-well plate to measure light absorbance at 555 nm. The result of experiment indicates after fibroblast cells are subjected to action of Aquilaria malaccensis seed oil of 50 and 100 μg/mL for 48 hours, secretion of collagen by the fibroblast cells can be effectively increased by 22-61%.

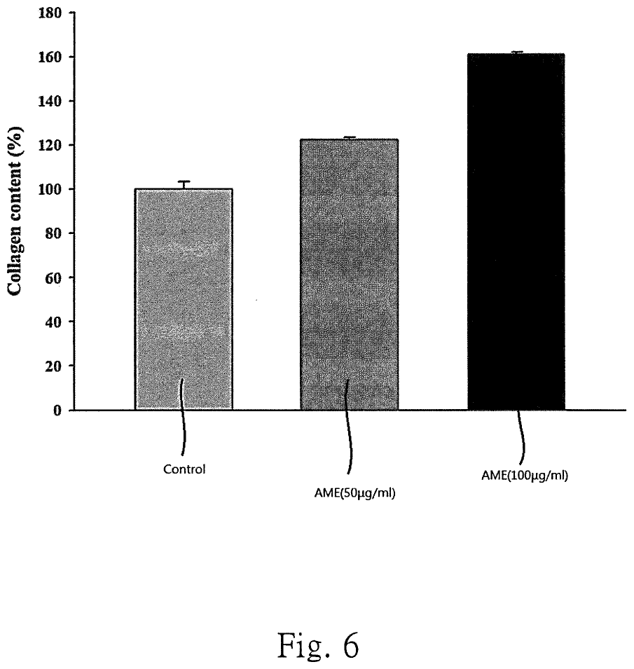

(2) Experiment results are provided in FIG. 6, which shows after fibroblast cells are subjected to action of Aquilaria malaccensis seed oil of 50 and 100 μg/mL for 48 hours, secretion of collagen by the fibroblast cells can be effectively increased by 22-61%.

### Experiment 4

The experiment is provided to inspect if the Aquilaria malaccensis seed oil can enhance wound healing.

(1) Placing an insert in a 24-well plate, cultivating cells of 3×10⁵ cell/mL in the insert for cultivation in an incubator of 37° C. and 5% CO₂ for at least 24 hours, removing the insert to proceed with irradiation of 50 mJ/cm² UVB, and then adding he Aquilaria malaccensis seed oil at different concentration s for reaction for 4 hours, and then observing with a microscope for the changes at 0, 12, 24, and 48 hour and taking photographs, and finally carrying out quantitative analysis.

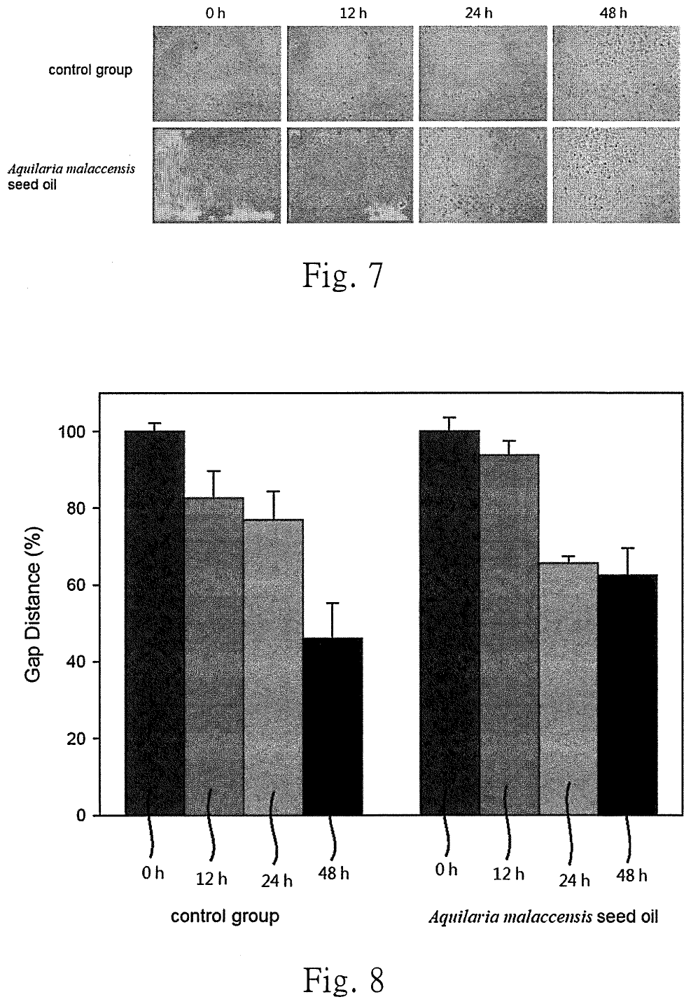

(2) Experiment results are provided in FIGS. 7 and 8, which show after being acted with the Aquilaria malaccensis seed, the ratio of gap distance of cells is observed at 0, 12, 24, and 48 hour, wherein the control group is 100.0%, 82.7%, 76.9%, and 46.1%, while the Aquilaria malaccensis seed oil is 100.0%, 93.8%, 65.6%, and 62.5%.

In view of the above experiments, it can be appreciated that the Aquilaria malaccensis seed extract (namely the above-described Aquilaria malaccensis seed oil) according to the present invention provides effects of protecting and repairing skin cells, enhancing generation of skin collagen, and enhancing healing of wounds. The use of the Aquilaria malaccensis seed extract (namely the above-described Aquilaria malaccensis seed oil) according to the present invention shows significant effects in skin protection and repair and anti-aging.

Further, to improve the application of the Aquilaria malaccensis seed extract (namely the above-described Aquilaria malaccensis seed oil) according to the present invention, the action concentration of the Aquilaria malaccensis seed extract (namely the above-described Aquilaria malaccensis seed oil) according to the present invention, which is 50-100 μg/mL (0.005-0.01%), as used in the above-described experiments can be magnified for 100 times, and the Aquilaria malaccensis seed extract (namely the above-described Aquilaria malaccensis seed oil) with the effective action concentration (0.5-1%) can be added with vitamin E that resists oxidation in an oil phase and olive oil and rose hip seed oil that function to moisturize skin, or can also be added with other components, such as hyaluronic acid that keeps skin wet and soft in aqueous phase, xanthan gum that functions for wet keeping and thickening, align that functions for skin smoothening and thickening, and chlorphenesin that function to improve preservation, so as to provide various products like compound Aquilaria malaccensis seed oil and Aquilaria malaccensis seed oil essence lotion. Thus, the Aquilaria malaccensis seed extract according to the present invention provides effects of anti-aging for skin, repair and treatment of damage cells, and enhancing healing of wounds, and as such, the present invention is usable for preparation of composition for skin care and repair.

I claim:

1. A use of Aquilaria malaccensis seed extract in preparation of skin care composition, wherein multiple Aquilaria malaccensis seeds are subjected to wash, in separate batches, with flowing reverse osmosis water; each wash is performed for 10 minutes; each batch of the Aquilaria malaccensis seeds is washed repeatedly for three times, and is then placed in room temperature for draining to dry, and is then spread and laid on a water-absorbing material for drying in shade for two days; and afterwards, a pulverizing device is used to pulverize the Aquilaria malaccensis seeds, such that the pulverized Aquilaria malaccensis seeds have a particle size that is not greater than 2 mm; and the pulverizing device is controlled in respect of pulverization time such that continuous pulverization is performed only for a time period not exceeding 20 minutes; next, the pulverized Aquilaria malaccensis seeds are subject to ultrasonic extraction, in separate batches, with 90% ethanol in such a way that each batch of the pulverized Aquilaria malaccensis seeds is subjected to extraction for three times, with only one extraction performed in each day; and after the completion of extraction, all ethanol-water solvent is removed by means of a rotary evaporator and a vacuum pump, so as to obtain Aquilaria malaccensis seed extract (Crude-EtOH), wherein the Aquilaria malaccensis seed extract (Crude-EtOH) is in an oily form and presented as an Aquilaria malaccensis seed oil.

2. The use of Aquilaria malaccensis seed extract in preparation of skin care composition according to claim 1, wherein an effective action concentration of the Aquilaria malaccensis seed extract is 50-100 μg/mL.
